(19) United States
(12) Patent Application Publication
(10) Pub. No.: US 2006/0264857 A1
(43) Pub. Date: Nov. 23, 2006

(54) Absorbent articles with buffer

(76) Inventor: Adrian Colbert, West Midlands (GB)

Correspondence Address: MYERS BIGEL SIBLEY & SAJOVEC, PO BOX 37428, RALEIGH, NC, 27627, US

(21) Appl. No.: 10/499,570

(22) PCT Filed: Dec. 20, 2002
(86) PCT No.: PCT/GB02/05829

§ 371 Date: Apr. 27, 2005

(30) Foreign Application Priority Data
Dec. 21, 2001 (GB) 0130666.1

(51) Int. Cl. A61F 13/20

(52) U.S. Cl. 604/360; 604/904

(57) ABSTRACT

A vaginal article is configured to balance the pH of vaginal mucosa and/or menses. There is also described a method of treating or alleviating or prophylactically preventing a vaginal disorder which involves the use of such an article.

The present invention relates to absorbent articles, e.g. tampons, wipes, etc. and methods of manufacture.

Vaginal problems can occur for a variety of reasons, inter alia, transfer of organisms from other body areas, e.g. anus, fingers, etc., from other people, i.e.; during sexual intercourse, from contact with materials, e.g. toilet seat, from irritation via textile materials, e.g.; panty gusset, or once a fungal infection is present, it may reappear if the conditions are favourable to re-present the original condition.

The vagina is home to a large number of microbes known as the normal flora. These organisms usually cause no illness in a healthy individual. Their presence protects the individual from foreign microorganisms and overgrowth of pre-existing organisms that can lead to pathogenic states. They do this by taking up available attachment sites and by producing antimicrobial compounds such as hydrogen peroxide and bacteriocin like substances (Boris s et al 2000).

Age related physiological changes affect the composition of the normal flora. Prior to puberty the conditions within the vagina favour the growth of species such as staphylococci, streptococci, diphtheroids and some coliforms (Talaro, K., et al 1996). A pH of 7 is normal in a pre-pubescent girl who would produce little oestrogen. At puberty production of oestrogen increases and induces secretion of glycogen from vaginal mucosal cells. Lactobacillus aerophilus then begins to predominate and ferment the glycogen resulting in the normal acid pH (3.8-4.2 (Plourd, D., M., 1997)) that prevents overgrowth by other commensuals. Nagy et al 1992 believed that the acidic pH helps to maintain the balance of the vagina by increasing the binding capacity of lactobacilli. At menopause the flora returns to a similar composition and therefore the pH that exists at prepuberty. There is a relationship between increased vaginal pH and abnormal vaginal secretions Poupas, A., et al 1985).

Vaginal thrush affects 20 million women each year. It is particularly common in women taking the contraceptive pill or antibiotics (particularly tetracycline), during pregnancy, and in diabetics. An increased hormone level in pregnant women and those taking oestrogen containing contraceptive pills raises vaginal pH. This provides an environment suitable for fungal growth and nourishment such as Candida albicans the causative agent of thrush. Candida albicans is a fungus that infects the mucous membranes of the vagina. Though the organism is a component of the normal commensuals population, an increase in its concentration can lead to the pathogenic condition known as thrush. Overgrowth can occur if vaginal pH rises, competing commensuals then diminish allowing the load of Candida spp. to increase substantially. It causes an uncomfortable vaginitis in those suffering from this condition. A vaginal discharge may occur accompanied by dysuria and urethritis.

Bacterial vaginosis was first reported in 1995 and it is currently the most prevalent cause of infectious vaginitis (Gonzalez Pedraza Aviles A et al,1999). Bacterial vaginal infections occur when the usually predominant lactobacillus population are replaced with a mixed predominantly anaerobic population including Gardnerella vaginalis a normal commensual of the vagina (Spiegal C., A., et al 1983), Bacterioides spp., Eubacte spp and mycoplasma (Hill G B et al 1984). These infections are common and are associated with a pH greater than 4.5 (Wang, J, 2000).

Treatment leading to dominance by Lactobacilli results in a lowered pH and therefore the absence of the bacterial infection. It is important to treat BV due to its association with a number of complications such as cervicitis, endometritis, pelvic inflammatory disease and a possible link with intraepithelial neoplasia (Georgijevic, A., et al 2000).

Conventional absorbent tampons such as catamenial tampons normally comprise an absorbent material of general cylindrical shape, and can be formed from a length of absorbent material comprising hydrophilic fibres such as cellulosic fibres by compressing the material longitudinally or laterally or both. Tampons formed by lateral compression and in particular radial compression have the advantage of being capable of expanding laterally in use thereby inhibiting the leakage of menses around the inserted tampon. A typical method of forming radially compressed tampons is given in British Patent No. 1082770.

The pH level of vaginal mucosa is prepubescent or postmenopausal females is generally around neutral i.e. pH 7. However, we have recently established that the pH of vaginal mucosa in a female may vary depending upon the stage of the menstrual cycle. In females experiencing the menstrual cycle the pH of the vaginal mucosa is generally from pH 4 to pH 4.5. However, since the pH of blood is generally higher than the pH of vaginal mucosa, during the period of menstruation, when the lining of the uterus is shed, the pH of the vagina. Thus, the pH of vaginal mucosa may increase. It is thought that this may be due to the presence of quantities of blood in the mucosa which will generally have a pH of 7.

However, this change in pH of the vaginal mucosa gives rise to a number of problems. Thus, for example, the change from a significantly acidic pH to a neutral pH means that a female can be prone to infections and/or other disorders.

It is also understood that HIV appears to survive best at a neutral pH, studies have shown that both cell-free HIV and HIV-infected cells are rapidly inactivated at pH levels below 4.5 and 5.5 respectively, as are several harmful bacteria including those causing gonorrhoea and bacterial vaginosis. Following unprotected vaginal intercourse, semen raises the pH of the vagina and keeps it elevated at around 5.5 to 7.0 for at least two hours. It is believed that this rise in pH facilitates the survival of sperm but also the survival of any HIV that may be present in the semen.

Following our discovery of these changes in the pH of vaginal mucosa, we have now developed a vaginal article e.g. a tampon which attempts to mitigate the problems of pH variation. Vaginal hygiene wipes are known, but these generally have a pH of greater than about 5.5.

Thus according to the invention we provide a vaginal article which is adapted to balance the pH of vaginal mucosa and/or menses.

The vaginal article may be a feminine wipe. However, preferably, the vaginal article of the invention may be an intra vaginal article, for example a tampon.

Thus, the article of the invention may be provided with a pH control agent. Preferentially, the pH control agent is an acid-buffering agent, which should be a non-toxic, non-irritating acidic material which release protons. A variety of acid-buffering agents may be used, preferably the acid-buffering agent is one or more low molecular weight organic or inorganic acids, and salts thereof, high molecular weight polymeric acids or ion exchange resins and fibres in the hydrogen form. Most preferably the acid-buffering agent is selected from the group including citric acid and sodium citrate. Preferably the pH control agent is one which reduces the pH to a range of from 3.5 to 5.5, preferably from 3.8 to 4.2 and most preferably to about pH 4.

During menstruation a conventionally tampon is worn for a period of from 4 to 8 hours. Furthermore, since, as hereinbefore described, following intercourse, vaginal pH is elevated for a period of about two hours, an especially preferred pH control agent is one which reduces the vaginal pH to the range described above, e.g. 3.5 to 5.5, and maintains that reduced vaginal pH for several hours. Preferably, the reduction in vaginal pH is maintained for at least 2 hours and more preferably for a range between 4 to 8 hours.

The acid-buffering agent may be in a variety of forms, such a hydrogel or a thin film. Certain hydrogels which may be mentioned are ACIDFORM and Aci-Jel, both of which are currently undergoing clinical trials.

Thus, the tampon may be provided with a coating of a pH control agent or, alternatively, the tampon of the invention may be impregnated with a pH control agent. Of course, it is within the scope of the present invention to provide a tampon which is impregnated, or partially impregnated and carries a coating of a pH control agent. In a yet further alternative the tampon of the invention may utilise a packaging system such as that disclosed in our co-pending application No. GB 0031655.4. Alternatively, the fibres of the tampon may be treated to render them pH balancing. Such treatment may, for example, include pre-treatment of the fibres prior to them being compressed into a tampon. In a further alternative, the nature of the fibres used may be selected such that they possess a pH balancing effect.

In a further embodiment of the invention the tampon may also be provided with a medicament. A variety of medicaments may be chosen. In particular, a group of antinycotic (antifungal) drugs known as imidazoles have been found to be effective in the treatment of Candida species. Four main drugs are used; clotrimazole, miconazole, econazole and ketoconazole. They work by blocking production of ergosterol, the main sterol in the fungal cell membrane. This ultimately effects the action of membrane-associated enzymes so that replication of the fungus is inhibited. It also prevents the non-pathogenic form of the microbe developing into the invasive form (hyphae). The drugs produce cell necrosis by inhibiting peroxidase enzymes. Development of resistance to the imidazoles is rare.

Clotrimazole interferes with amino acid transport into the organism by attacking the cell membrane. It is a drug of choice for treatment of candidiasis of the vagina. Adverse effects can occur on application to the skin and include stinging, erythema, pruritis, peeling. The incidence however is low. Intravaginal administration can be associated with a burning sensation and lower abdominal cramps. This drug is available as a cream or tablet for intravaginal use and can also be applied topically.

Miconazole is used topically, but rarely systemically due to toxicity issues. Systemic use is by intravenous infusion and is associated with fever, nausea, and a rash. It also can enhance the action of oral anticoagulants leading to haemorrhage. Around 15% of C. albicans are resistant to clotrimazole and miconazole. Recurrent infections can be treated with fluconazole.

Econazole is available for topical application. Adverse effects include burning and itching sensations, though these have -been observed in just 3% of users. Less than 1% of topical econazole is absorbed. A 1% cream is applied twice a day for 2 wweeks.

Ketoconazole can be administered orally to treat superficial mycoses. The toxicity associated with the drug means that its' use is only advised for those mycoses which have not responded to topical agents.

Other treatments include Nystatin, a polyene antibiotic. It is used solely to treat candidiasis by topical, intravaginal and oral routes. Significant absorption has not been observed with any of these routes. Intravaginal application of the drug is by administration of a vaginal tablet. Dosage ranges between 100,000-200,000 units daily for a period of two weeks. Adverse effects reported by Lehne et al are limited to oral Nystatin (occasionally causes gastrointestinal disturbances) and topical application (can cause local irritation).

Treatment of BV usually consists of metronidazole or clindamycin by mouth or intravaginally (Georgijevic A., et al 2000) (Wang, J 2000) (Gonzalez Pedraza Aviles A., et al 1999). Hanson et al compared metronidazole in two forms in 2000. Two groups were involved in the trial, one was treated with an oral dose of metronidazole and the other group treated with metronidazole vaginal gel. The efficacy in both treatment groups was comparable. The application of 0.75% vaginal gel twice daily for 5 days was associated with fewer gastrointestinal complaints than standard oral treatment. In the case of recurrent bacterial vaginosis, Winceslaus et al (1996) found that a single vaginal washout with 3% hydrogen peroxidase cleared symptoms of 78% of those involved in the trial.

Liposomes made of phosphatidylcholine have been tested for their ability to act as a carrier for drug delivery to the vagina in the treatment of vaginal infections. In vitro and in situ studies carried out by Pavelic et al confirmed the ability of liposomes to act as a carrier system for vaginal delivery.

A new vaginal formulation called ACIDFORM has been designed as an antimicrobial/contraceptive product. It is acid-buffering and can therefore maintain the acidic environment of the vagina (McLean N., W., et al 2000). Garg et al found ACIDFORM to be more effective and bioadhesive than Aci-Jel (a commercial acid buffering vaginal product)

Another option of treating vaginal bacteriosis is to use Lactobacillus isolates to recolonise the vagina and therefore lower and maintain the acidic pH McLean, N., W., et al 2000).

In a yet further aspect of the invention the tampon may also be provided with a lubricant, e.g. in the form of a coating, for example, on a shoulder portion of the tampon, provided it is sufficient to allow insertion of the tampon into the vagina without undue discomfort.

The lubricants used in the invention can be any of the lubricants which are suitable for coating absorbent tampons to reduce the frictional drag thereof during insertion into the vagina.

Preferred lubricants for use in the invention comprise a water soluble polymer. Such a water soluble polymer lubricant coating on a tampon of the invention in use can, on contact with the vagina wall, absorbed moisture and become highly lubricious thus further enhancing the lubricant nature of the coating. Furthermore a water soluble polymer lubricant coating on a tampon after insertion may eventually be dissolved by fluids present in the vagina from the surface of the tampon thereby making the surface available for absorbing menses.

The absorbent tampon used in the invention can be any of the absorbent tampons conventionally used for catamenial purposes which has a rounded insertion end and is capable of expanding laterally in use. Suitable methods of forming a rounded end on a absorbent include those disclosed in British Patent No. 1046066. The rounded insertion end can be hemispherical, rounded conical or alike rounded tapered shape. Favoured tampons of the invention comprise a spirally wound fleece of absorbent cellulosic fibres which has been compressed radially for example between compression dies.

Suitable methods of making tampons comprising spirally wound fleece include those disclosed in British Patent No. 1098400. Suitable methods of forming tampons by radially compressing absorbent material include those disclosed in British Patent 1082770.

The absorbent tampons of the invention will be suitable for digital use. The tampons can be packed within a wrapper of water impermeable film such as moisture proof coated cellulose film, polypropylene film or polypropylene film. The film can conventionally be closed by a breakable seal for example a heat seal over insertion end to enable the tampon to be inserted via the seal from the wrapper into the vagina. Suitable wrappers of this type are disclosed in British Patent No. 1305472. Alternatively, as previously mentioned, a tampon, which may optionally be uncoated and/or unimpregnated, may utilise a packaging system such as that disclosed in our co-pending application No. PCT/GB01/05622.

The tampon within the wrapper can favourably be sterile. Suitable methods of sterilising include gamma and electron beam irradiation methods.

The materials used in the method of the invention can be the materials described hereinbefore in relation to the tampon of the invention.

In the method of the invention pH control agent may be provided on the tampon by any suitable method. Suitable methods include, but shall not be limited to, adhering a preformed solid film of the lubricant to the tampon by for example solvent or heat and coating the lubricant in liquid form for example as a hot melt or as a solution onto the tampon by spraying or other coating method.

In a method of the invention which comprises spraying, the lubricant can be sprayed as a band on the shoulder portion of the insertion of the tampon by controlling the width of the spray and rotating the spray head or the tampon under the spray head. In such methods the surface of the main body of the tampon which is adjacent to the shoulder portion or region can advantageously be surrounded by a cylindrical member to inhibit the lubricant being coated on the side surface of the tampon. The cylindrical member can favourably be part of the outer wrap of the tampon before it is sealed.

A wide variety of non-toxic, non-irritating acidic materials which release protons can serve as pH control agents. For instance, these materials can be low molecular weight organic or inorganic acids, high molecular weight polymeric acids or ion exchange resins and fibres in the hydrogen form. Particular pH control agents which may be mentioned include citric acid and sodium citrate.

The deposition of the active ingredient of the pH control agent onto the skin of the user can occur both through dry and/or wet transfer within the absorbent article environment.

The absorbent article of the present invention can include the pH control agent in at least a portion of any of its interior component parts in an amount sufficient to maintain prolonged natural skin pH.

It is particularly preferred that a component part of the absorbent article which comes into substantial contact with the vaginal mucosa of the intended user includes the pH control agent. In another preferred embodiment, the pH control agent can be included in at least a portion of the absorbent core and at least a portion of the topsheet or tissue layer, although any single component part or combination of component parts is within the scope of the present invention.

Preferably, the pH control agent is present in an amount of at least about 1% by weight, preferably from about 1% by weight to about 10% by weight, and more preferably about 2% by weight of the pH control agent, based on the total weight of the tampon. In a particular embodiment, which may be mentioned, the pH control agent may comprise about 2% by weight citric acid, based on the total weight of the tampon.

According to a further aspect of the invention we provide a method of manufacturing a tampon as hereinbefore described characterised in that the method comprises adhering, coating or impregnating a pH control agent onto or into a vaginal article.

A variety of processes may be used in the method of the invention. Thus, in one aspect, a preformed solid film of an acid buffer may be applied to the tampon by, for example, solvent or heat. Alternatively, the acid buffer may be applied in liquid form, as a hot melt or as a solution onto the tampon by spraying or other coating method, including, for example, kiss-coating.

One method which may be mentioned is that of spraying the acid buffer as a hot melt onto the shoulder area of the tampon.

Preferably, the pH control agent is applied to the treated portion of the component part of the absorbent article as an aqueous solution comprising the pH control agent.

In another aspect the invention provides a method of treating or alleviating or prophylactically preventing a vaginal disorder which comprises the use of a vaginal article as hereinbefore described.

Thus the use of the article of the invention, and especially the tampon of the invention may, in lowering vaginal pH, also act as a spermicidal agent and/or a means of preventing or alleviating transmission of HIV. Therefore according to a further aspect of the invention we provide a method of treating, alleviating or preventing the transmission of HIV which comprises the use of a tampon of the invention.

The use of the tampon of the invention may also be advantageous as a means of preventing or alleviating the transmission of other sexually transmitted diseases. Such diseases may include, but shall not be limited to those resulting from a viral disease, a bacterial disease, a protozoal disease and/or a fungal disease. When the disease is a viral disease, it may be selected from HIV and genital herpes. When the STD is a fungal disease, it may be, for example, Candida, e.g. Candida albicans. In a further alternative aspect of the invention, the STD may be a bacterial disease, such as chlamydia.

Thus, specific STDs, which may be mentioned, are bacterial vaginosis, chlamydia, genital herpes, genital warts, gonorrhoea, syphilis, trichomoniasis and Candida.

In the method of the invention a tampon may be used alone or in conjunction with a hygienic wipe as hereinbefore described.

Thus according to a further aspect of the invention we provide a kit comprising a wipe and a tampon as hereinbefore described.

The invention will now be illustrated by way of example only.

### EXAMPLE 1

| | | | RAW MATERIAL | |
|---|---|---|---|---|
| ATO: | INCI ADOPTED NAME: | RAW MATERIAL: | FUNCTION: | PERCENT: |
| ATO000 | Aqua (100%) | PURIFIED WATER | Vehicle | 75.495000 |
| ATO631 | Propylene Glycol (100%) | MONOPROPYLENE GLYCOL | Humectant/Solvent | 1.000000 |
| ATO513 | Sodium Methylparaben (100%) | NIPAGIN M SODIUM | Preservative | 0.100000 |
| ATO552 | Sodium Propylparaben (100%) | NIPASOL M SODIUM | Preservative | 0.040000 |
| ATO342 | Glycerin (100%) | PRICERINE 9091/ | Humectant | 20.000000 |
| | | GLYCERIN BP/USP/ | | |
| | | GLYCERIN (VEG) | | |
| ATO761 | Sodium Citrate (100%) | TRISODIUM CITRATE | Buffering Agent | 0.500000 |
| ATO544 | Disodium EDTA(100%) | VERSENE NA2 CRYSTALS/ | Chelating Agent | 0.150000 |
| | | NERVANAID BA2 POWDER | | |
| ATO555 | Hydroxyethylcellulose(100%) | NATROSOL 250 HX-PHARM | Thickener | 2.133000 |
| AT0143 | 2-Bromo-2-Nitropropane-1,3-Diol (100%) | MYACIDE PHARMA BP/BRONOPOL | Preservative | 0.025000 |
| AT0185 | Citric Acid (100%) | CITRIC ACID | pH Adjuster | 0.557000 |
| | | | TOTAL | 100.0000 |

### EXAMPLE 2

| | | | RAW MATERIAL | |
|---|---|---|---|---|
| ATO: | INCI ADOPTED NAME: | RAW MATERIAL: | FUNCTION: | PERCENT: |
| ATO000 | Aqua (100%) | PURIFIED WATER | Vehicle | 75.495000 |
| ATO631 | Propylene Glycol (100%) | MONOPROPYLENE GLYCOL | Humectant/Solvent | 1.000000 |
| ATO513 | Sodium Methylparaben (100%) | NIPAGIN M SODIUM | Preservative | 0.100000 |
| ATO552 | Sodium Propylparaben (100%) | NIPASOL M SODIUM | Preservative | 0.040000 |
| ATO342 | Glycerin (100%) | PRICERINE 9091/GLYCERIN | Humectant | 20.000000 |
| | | BP/USP/GLYCERIN (VEG) | | |
| ATO463 | Lactic Acid (88%), Aqua (12%) | PURAC SP88 | Humectant | 0.390400 |
| D0317 | Sodium Lactate (50%), Aqua (50%) | Sodium Lactate | | 0.666600 |
| ATO544 | Disodium EDTA (100%) | VERSENE NA2 CRYSTALS/ | Chelating Agent | 0.150000 |
| | | NERVANAID BA2 POWDER | | |
| ATO555 | Hydroxyethylcellulose (100%) | NATROSOL 250 HX-PHARM | Thickener | 2.133000 |
| ATO143 | 2-Bromo-2-Nitropropane-1,3-Diol (100%) | MYACIDE PHARMA BP/BRONOPOL | Preservative | 0.025000 |
| | | | TOTAL | 100.0000 |

1. A vaginal absorbent article which is adapted to balance the pH of vaginal mucosa and/or menses, the vaginal absorbent article comprising
a pH control agent which reduces the pH to a range of from about pH 3.5 to pH 5.5 and wherein the pH control agent maintains the reduced vaginal pH for several hours.

2. A vaginal absorbent article according to claim 1 wherein the article is a wipe.

3. A vaginal absorbent article according to claim 1 wherein the article is a tampon.

4. A vaginal absorbent article according to claim 1 wherein the pH control agent reduces the pH to a range of about 3.8 to 4.2.

5. A vaginal absorbent article according to claim 1 wherein the pH control agent reduces the pH to about pH 4.

6. A vaginal absorbent article according to claim 1 wherein the pH control agent maintains the reduced vaginal pH for at least 2 hours.

7. A vaginal absorbent article according to claim 1 wherein the pH control agent maintains the reduced vaginal pH for a range between about 4 to 8 hours.

8. A vaginal absorbent article according to claim 1 wherein the pH control agent comprises an acid-buffering agent

9. A vaginal absorbent article according to claim 8 wherein the acid-buffering agent comprises a non-toxic, non-irritating acidic material which releases protons.

10. A vaginal absorbent article according to claim 9 wherein the acid-buffering agent comprises at least one low molecular weight organic or inorganic acids and salts thereof, high molecular weight polymeric acids or ion exchange resins and fibres in the hydrogen form.

11. A vaginal absorbent article according to claim 10 wherein the acid-buffering agent is selected from the group consisting of citric acid and sodium citrate.

12. A vaginal absorbent article according to claim 8 wherein the acid-buffering agent is in the form of a hydrogel.

13. A vaginal absorbent article according to claim 12 wherein the acid-buffering agent is selected from the group consisting of ACIDFORM and Aci-Jel.

14. A vaginal absorbent article according to claim 1 wherein the article comprises a coating of the pH control agent.

15. A vaginal absorbent article according to claim 1 wherein the article is impregnated with the pH control agent.

16. A vaginal absorbent article according to claim 1 wherein the article comprises a medicament.

17. A vaginal absorbent article according to claim 16 wherein the medicament is antimycotic.

18. A vaginal absorbent article according to claim 17 wherein the antimycotic is an imidazole.

19. A vaginal absorbent article according to claim 18 wherein the imidazole is selected from the group consisting of clotrimazole, miconazole, fluconazole, econazole, metronidazole and ketoconazole.

20. A vaginal absorbent article according to claim 19 wherein the medicament is selected from the group consisting of metronidazole and clindamycin.

21. A vaginal absorbent article according to claim 1 wherein the article comprises a lubricant.

22. A vaginal absorbent article according to claim 1 wherein the weight per unit area of the pH control agent on or in the article is about 0.01 to 0.25 g/cm².

23. A vaginal absorbent article according to claim 1 wherein the article is packed within a wrapper of impermeable film.

24. A vaginal absorbent article according to claim 23 wherein the impermeable film is selected from the group consisting of a coated cellulose film, polypropylene film and polypropylene film.

25. A vaginal absorbent article according to claim 1 wherein the article is housed in a packaging system.

26. A vaginal absorbent article according to claim 1 wherein the pH control agent is in at least a portion of an interior component part of the article.

27. A vaginal absorbent article according to claim 1 wherein the pH control agent is present in the article in an amount of from about 1% w/w to 10% w/w.

28. A method of treating or alleviating or prophylactically preventing a vaginal disorder comprising:
using a vaginal absorbent article that comprises a pH control agent which reduces vaginal mucosa and/or menses pH to a range of from about pH 3.5 to pH 5.5 and wherein the pH control agent maintains the reduced vaginal pH for several hours.

29. A method of treating, alleviating or preventing the transmission of HIV comprising:
using a vaginal absorbent article that comprises a pH control agent which reduces vaginal mucosa and/or menses pH to a range of from about pH 3.5 to pH 5.5 and wherein the pH control agent maintains the reduced vaginal pH for several hours.

30. A method according to claim 29 wherein the article is a tampon.

31. A method according to claim 28 wherein the disorder is a sexually transmitted disease.

32. A method according to claim 31 wherein the sexually transmitted disease is selected from the group consisting of a viral disease, a bacterial disease, a protozoal disease and a fungal disease.

33. A method according to claim 32 wherein the sexually transmitted disease is selected from the group consisting of bacterial vaginosis, chlamydia, genital herpes, genital warts, gonorrhoea, syphilis, trichomoniasis and Candida.

34. A method according to claim 30 wherein the tampon is used in conjunction with a hygienic wipe.

35. (canceled)

36. A method of manufacturing an article, comprising:
applying a pH control agent to the article which reduces vaginal mucosa and/or menses pH to a range of from about pH 3.5 to pH 5.5 and wherein the pH control agent maintains the reduced vaginal pH for several hours.

37. A method according to claim 36 further comprising:
spraying an acid buffer as a hot melt onto a shoulder area of the article.

38. (canceled)
